(19) United States
(12) Patent Application Publication
(10) Pub. No.: US 2013/0338078 A1
(43) Pub. Date: Dec. 19, 2013

(54) SKIN CARE FORMULATIONS INCLUDING OCTAPEPTIDE COMPLEXES AND METHODS FOR THEIR MANUFACTURE

(71) Applicant: Alyson GALDERISI, Wayne, NJ (US)

(72) Inventor: Alyson GALDERISI, Wayne, NJ (US)

(73) Assignee: INTERNATIONAL EDGE. INC.,, Fairfield, NJ (US)

(21) Appl. No.: 13/524,031

(22) Filed: Jun. 15, 2012

(51) Int. Cl. A61K 8/64; A61Q 19/08

(52) U.S. Cl. 514/18.8

(57) ABSTRACT

The present invention relates to skin care formulations including octapeptide complexes. A preferred embodiment of the skin care formulation is a multi-active serum, which treats numerous skin problems associated with environmental exposure and aging. In one aspect, the multi-serum may be formulated for treatment of facial skin, and may optionally be applied as a mask. In another aspect, the multi-active serum may be formulated specifically for the treatment of delicate skin in the under-eye area of the fact. This embodiment of the invention may be applied as an under eye mask. In yet another aspect, the invention provides a method of manufacturing skin care formulations including octapeptide complexes.

### FIELD OF THE INVENTION

The present invention relates to skin care formulations including octapeptide complexes for the treatment of visual signs of aging and environmental exposure, as well as methods of making such formulations.

### BACKGROUND OF INVENTION

Skin products such as soaps, gels, exfoliating scrubs, lotions and creams are widely available to care for the skin by cleansing, moisturizing and treating it. Users of skin products often suffer from environmental exposure and visible signs of aging such as wrinkles. Typically, users must apply separate products to treat each of their skin ailments. Additionally, most moisturizers and anti-aging products are in the form of lotions or creams, and thus contain binders and emulsifiers that can reduce the effectiveness of active ingredients and impair the skin's ability to heal and rejuvenate itself. Accordingly, there is a need for a multi-active serum that is effective against numerous skin ailments and avoids the use of binders and emulsifiers.

### SUMMARY OF INVENTION

The present invention provides a multi-active serum that includes clinically effective ingredients for the treatment of skin dryness, fine lines, deep wrinkles and hyperpigmentation, among other visible signs of poor skin health and aging. As such, it includes at least a gentle exfoliating agent, an anti-wrinkling agent and an anti-inflammatory agent. These agents work synergistically to support the skin's natural ability to repair itself, which is known to decline with age and with overexposure to the elements. The multi-active serum may be applied the entire facial area, or may be specifically formulated to treat the under-eye area. Additional ingredients may be included to complement and augment the primary serum ingredients' functions.

In general, in one aspect, the invention features a skin care formulation for topical application to human skin including an exfoliating agent including a plant-based β-hydroxy acid, a muscle contraction-inhibiting agent including an effective amount of acetyl octapeptide-3, and an anti-inflammatory agent including glycosaminoglycans.

The invention may also include one or more of the following features. The skin care formulation may be a serum. The skin care formulation may include a lipid barrier-building agent including one or more ceramides. The skin care formulation may include a tissue-restructuring agent including yeast extract in an aqueous solution of sorbitol. The skin care formulation may include a skin repair-activating agent including palmitoyl oligopeptide & palmitoyl-tetrapeptide-7, and a skin texture-enhancer including tripeptide-1. The skin care formulation may include an enzyme-inhibiting agent including purified rice peptides. The skin care formulation may include glycyrrhiza glabra extract, ahnfeltia concinna extract and glycerin.

A mask for the treatment of visible signs of aging may include a physiologically acceptable fabric saturated with the serum of the present invention. The mask may be configured to cover a human face or the under-eye areas of a human face.

The exfoliating agent may include willow bark extract. The skin care formulation may include up to 20% by weight of the exfoliating agent or up to 15% by weight of the exfoliating agent. The skin care formulation may include up to 10% by weight of the muscle contraction-inhibiting agent or up to 5% by weight of the muscle contraction-inhibiting agent. The skin care formulation may include up to 10% by weight of the anti-inflammatory agent or up to 5% by weight of the anti-inflammatory agent. The skin care formulation may include between 1% and 10% by weight of the exfoliating agent or up to 3% by weight of the anti-inflammatory agent.

### DETAILED DESCRIPTION OF THE INVENTION

The present invention provides skin care formulations that moisturize dry skin and treat several of the visible signs of aging, including fine lines and wrinkles, hyperpigmentation and loss of elasticity.

According to a first embodiment, the skin care formulation is an aqueous, multi-active serum having effective amounts of a gentle exfoliating agent, an anti-wrinkling agent and an anti-inflammatory agent. The gentle exfoliating agent may be a beta-hydroxy acid, and in particular may be a plant-derived beta-hydroxy acid, such as that found in willow (Salix Nigra) bark extract. The anti-wrinkling agent may be embodied by an octapeptide such as the acetyl octapeptide-3 used in the snap-8® complex. The anti-inflammatory agent may be embodied in a component such as MDI Complex®, which includes glycosaminoglycans.

In another embodiment, the skin care formulation may also include a component that mimics the lipid barrier layer of youthful, healthy skin and that augments the capacity of older or stressed skin to maintain itself. This component may include ceramides, phytosphingosine, sodium lauroyl lactylate, carbomer and xanthan gum. The ceramides may include ceramide 3, ceramide 3 II, ceramide 6 and ceramide 1. The commercially available SK-Influx® product would provide these beneficial ceramides.

In yet another embodiment, the skin care formulation may include an enzyme-inhibiting agent including rice peptides. The enzyme-inhibiting agent may be an active complex such as REGU®-AGE, which includes hydrolyzed rice protein, soy peptides and biotechnologically produced yeast protein.

Other components that may be combined with the gentle exfoliating agent, anti-wrinkling agent and anti-inflammatory agent include glycerin, Matrixyl 3000® complex which includes pentapeptides, Kollaren® complex which includes tripeptide-1, Drieline® complex which includes sorbitol and yeast extract, APT® complex which includes Ahnfeltia Concinna extract, and Net-DG® complex which includes Glycyrrhiza (Licorice Risome) Glabra. Suitable preservatives such as phenoxyethanol and Symdiol 68® complex which includes 1,2-hexandiol, caprylyl glycol and tropolone, also may be included.

Multi-Active Serum

Exfoliating Agent—Willow bark extract, which contains salicin, provides natural beta-hydroxy activity without causing the undue skin irritation commonly associated with synthetic salicylic acid. Like alpha-hydroxy acids, beta-hydroxy acids work as keratolytics by accelerating skin cell turnover and allowing room for new cell growth. However, because the molecules of beta-hydroxy acids are larger than the molecules of alpha-hydroxy acids, beta-hydroxy acids penetrate the skin more slowly. This slower penetration affects the sloughing action of the beta-hydroxy acid so as not to outpace the skin's capacity to renew itself, which minimizes redness and irritation.

Anti-Wrinkling Agent—Wrinkling of the facial skin is usually caused by histological, biochemical and physiological changes in aging tissue, which are enhanced by environmental exposure. Repeated facial movements caused by facial muscle activation can disturb the ideal conformation of the skin's lipid matrix, which contributes to skin wrinkling.

As with all muscles, facial muscles contract when musculoskeletal nerves receive neurotransmitter molecules. The increased presence of active soluble N-ethylmaleimide-sensitive attachment receptor protein (SNARE) complexes increases the release of neurotransmitter molecules at muscle fiber synapses. Because acetyl octapeptide-3 antagonistically competes with a native receptor protein for positions in SNARE complexes, SNARE complex activity is reduced, and muscle contraction is inhibited. This inhibition of facial muscle contractions in turn reduces wrinkling of the skin. Acetyl octapeptide-3's activity and effectiveness is evidenced by the inhibited release of catecholamines and glutamate in vivo. The acetyl octapeptide-3 in the snap-8® complex therefore provides a safer and less expensive alternative to the muscle cell-disabling Botulinum Toxin, and one which may be incorporated into emulsions and sera for ease of application.

Anti-inflammatory Agent—Skin cells possess an extracellular matrix of structural carbohydrates and proteins that provides a structural foundation and supports tissue growth and wound healing. Matrix metalloproteinase (MMP) enzymes, which are activated by environmental stress and UV exposure, destroy the collagen that forms an essential part of the extracellular matrix. This adverse effect is experienced as inflammation of skin tissue. Glycosaminoglycans augment the skin's capacity to maintain the structural integrity of skin cells by inhibiting MMP enzymes. MDI Complex®, which includes glycosaminoglycans, is clinically effective at reducing the appearance of facial spider veins, rosacea and other irritations that arise when the extracellular matrix is compromised.

Other Agents—In addition to these primary ingredients, a number of additional beneficial compounds may be included to enhance the effectiveness of the skin care formulation.

Kollaren® complex is a skin texture-enhancer that includes tripeptide-1, which complements the activity of glycosaminoglycans by stimulating the production of extracellular matrix components. The inclusion of tripeptide-1 therefore supports the tissue renewal and healing functions of the extracellular matrix.

Drieline® complex is a tissue restructuring agent including yeast extract in an aqueous solution of sorbitol. This yeast extract is an advanced polysaccharide extracted from baker's yeast (Sacchromyces Cerevisiae) that works in synergy with the peptides mentioned above to accelerate skin repair by stimulating collagen synthesis, protecting against UV radiation, and otherwise reinforcing the skin's natural defenses.

Mask for Application to the Face

In one embodiment of the invention, the multi-active serum is applied to the face by means of a fabric mask. The serum according to the face-mask embodiment would include certain compounds that particularly complement facial skin.

The stratum corneum is the outermost lower of the skin. Living skin cells, such as keratinocytes, proliferate at the basal layer and migrate through the epidermis to the stratum corneum. The stratum corneum is made up of corneocytes or dead skin cells, which form a protective barrier for the underlying tissue. During cornification, that is when living keratinocytes become non-living corneocytes, the lipid-containing cell membrane is infiltrated by ceramides, which covalently link to structural proteins forming a resilient envelope. When such ceramides are insufficient, the lipid barrier layer of the stratum corneum is compromised. Accordingly, in one embodiment, the serum includes ceramides such as those found in the SK-Influx® complex work to replenish the lipid barrier of the skin, resulting in enhanced moisturization and protection.

The serum may also include Matrixyl 3000® complex, which is a skin repair-activating agent that includes palmitoyl oligopeptide (Pal-glycine-histidine-lysine, hereinafter “Pal-GHK”) and palmitoyl-tetrapeptide-7 (Pal-glycine-glutamate-proine-arginine, hereinafter “Pal-GQPR”). Pal-GHK and: Pal-GQPR are engineered peptides connected to palmitic acid, which is a fatty acid that improves both peptides' lipid solubility thereby allowing the peptides to penetrate the skin more readily. The GHK peptide is a fragment of the type I collagen molecule which, when present, signals the skin's matrix-producing fibroblasts that more matrix materials are needed. The GQPR peptide reduces the production of interleukin-6 (IL-6) by keratinocytes and fibroblasts. IL-6 promotes inflammation, which leads to degradation of the extracellular matrix. By reducing IL-6, palmitoyl tetrapeptide-7 slows down the wrinkling and loss of elasticity that accompany injury to the extracellular matrix. Pal-GHK and Pal-GQPR work synergistically to augment the skin's ability to heal itself.

Viscarin SD 389® complex, which includes carrageenan, is a suitable carrier for the numerous active compounds of the serum, and, when included, enhances the skin's ability to retain moisture.

Mask for Application to the Under-Eye Area

The skin around the eyes is very thin and delicate, making it particularly susceptible to free radicals and UV radiation. Additionally, aging skin may suffer from poor hemodynamics and limited blood flow, which leads to increased blood retention and venous pressure in an area where the connective tissue is already weakened. The result is the appearance of dark circles and puffiness. Accordingly, the serum may include hydrolyzed rice protein, such as that provided in the REGU®-AGE complex, which inhibits enzyme activity caused by UV radiation. Hydrolyzed rice protein also exerts a localized hemodynamic effect thereby improving the supply of oxygen to the tissue. The oxidoreductase included in the REGU®-AGE complex minimizes free radicals, which in turn minimizes inflammation processes. These combined properties visibly improve the appearance of dark circles and puffiness and retard the skin degeneration that leads to wrinkles and slackness.

The serum may also include the Net-DG® complex, which includes dipotassium glycyrrhizinate that is derived from licorice (glycyrrhiza glabra) rhizome or root. Licorice root extract is an anti-inflammatory ingredient that inhibits the release of histamine and enhances hyaluronic acid retention in the skin cells, acts as an emollient and humectant, and is an antioxidant. This gentle, naturally-derived product is therefore particularly useful for treatment of the delicate under-eye area.

The serum may also include the APT® complex, which includes Ahnfeltia Concinna extract. This extract is produced by biofermentation in the cells of a strain of red marine algae. Ahnfeltia Concinna extract is a gel-forming skin protector that provides high concentrations of essential amino acids to the skin. The hydroxyproline of the APT® complex is thought to signal to skin cells that breakdown of collagen has occurred and needs to be repaired. In combination with the other active ingredients discussed, APT® complex substantially improves skin smoothness and firmness.

### EXAMPLE 1

An embodiment of the invention may include up to 20% by weight of willow bark extract solution, up to 10% by weight of an acetyl octapeptide-3 complex, and up to 10% by weight of a glycosaminoglycans complex. In a preferred embodiment, the serum includes 15% by weight of carruba 10% quantitated willow bark extract, 5% by weight of the snap-8® complex, and 5% by weight of the MDI Complex®. The following formulation is preferably used in a mask for the face.

TABLE 1

| | | | % |
|---|---|---|---|
| Seq. | INCI Name | Trade Name | W/W |
| 1 | Water (Aqua) | Water | 63.00 |
| 2 | Carrageenan (Chondrus | Viscarin SD 389 (FMC) | 1.00 |
| | Crispus) | | |
| 3 | Salix Nigra (Willow) | Willow Bark Extract | 36.00 |
| | Bark Extract | (Carrubba 10% Quantitated) | |
| | Glycerin; | Matrixyl 3000 (Sederma) | |
| | Butylene Glycol; | | |
| | Water (Aqua); | | |
| | Carbomer; | | |
| | Polysorbate-20; | | |
| | Palmitoyl-Pentapeptide 3 | | |
| | Glycosaminoglycans | MDI Complex (Atrium/TAOS) | |
| | Ceramide 3; | Sk-Influx (Centerchem) | |
| | Ceramide 3 II; | | |
| | Ceramide 6; | | |
| | Ceramide 1; | | |
| | Cholesterol; | | |
| | Phytosphingosine; | | |
| | Sodium Lauryl Lactylate; | | |
| | Carbomer; | | |
| | Xanthan Gum | | |
| | Phenoxyethanol | Phenoxyethanol | |
| | Tripeptide-1 | Kollaren (Atrium/Taos) | |
| | Sorbitol; Yeast Extract | Drieline (Atrium/TAOS) | |
| | Acetyl Octapeptide-3 | Snap 8 (Centerchem) | |

### PREPARATION OF EXAMPLE 1

In a stainless steel kettle equipped with a LIGHTNIN® mixer, add Seq. 1 and heat to 80° C. under LIGHTNIN® mixing, just long enough to kill any potential gram positive bacteria. Cool the water to 70° C. At 70° C. begin adding Seq. 2. The carrageenan of Seq. 2 should be added slowly to make sure it is evenly dispersed and no lumps are present. Samples should be taken from the bottom and top of the liquid in the kettle to be quality-checked before proceeding. Cool the resulting mixture to 35° C. Add the components of Seq. 3 one at a time, ensuring that each ingredient is evenly dispersed before proceeding. Mix until uniformity is obtained and compare to standard. Specifications of the standard may be determined by the skilled artisan after five production units of the serum are examined. A physiologically acceptable fabric configured to substantially cover a human face may be saturated with the prepared serum to facilitate delivery of the effective ingredients to the user's skin.

### EXAMPLE 2

Another embodiment of the invention may include up to 5% by weight of willow bark extract solution, up to 10% by weight of an acetyl octapeptide-3 complex, and up to 10% by weight of a glycosaminoglycans complex. In a preferred embodiment, the serum includes 2% by weight of carruba 10% quantitated willow bark extract, 5% by weight of the snap-8® complex, and 3% by weight of the MDI Complex®. The following formulation is preferably used in a mask for the under-eye area.

TABLE 2

| | | | % |
|---|---|---|---|
| Seq. | INCI Name | Trade Name | W/W |
| 1 | Water (Aqua) | Water | 71.00 |
| 2 | Glycerin | Glycerin | 29.00 |
| | Salix Nigra (Willow) | Willow Bark Extract | |
| | Bark Extract | (Carrubba 10% Quantitated) | |
| | Hydrolyzed Rice Bran | Regu-age (Centerchem) | |
| | Protein | | |
| | Glycosaminoglycans | MDI Complex (Atrium/TAOS) | |
| | Ahnfeltia Concinna | APT (Centerchem) | |
| | Extract | | |
| | Glycyrrhiza (Licorice | Net -DG (Barnet) | |
| | Risome/Root) Glabra | | |
| | 1,2-Henandiol Caprylyl | Symdiol 68 | |
| | Glycol; Tropolone | | |
| | Tripeptide-1 | Kollaren (Atrium/TAOS) | |
| | Sorbitol; Yeast Extract | Drieline (Atrium/TAOS) | |
| | Acetyl Octapeptide-3 | Snap 8 (Centerchem) | |

### PREPARATION OF EXAMPLE 2

In a stainless steel kettle equipped with a LIGHTNIN® mixer add Seq. 1 and heat to 80° C. under LIGHTNIN® mixing, just long enough to kill any potential gram positive bacteria. Cool the water to 45° C. At 45° C. add Seq. 2 materials one at a time, mixing to uniformity with each addition. Cool the uniform mixture to 25° C. Mix again until uniformity is obtained. Samples should be taken from the bottom and top of the liquid in the kettle to be quality-checked against a standard. Specifications of the standard may be determined by the skilled artisan after five production units of the serum are examined. A physiologically acceptable fabric configured to cover the under-eye areas of a human face may be saturated with the prepared serum to facilitate delivery of the effective ingredients to the user's skin.

Thus, there has been shown and described a skin care formulation and method of producing a skin care formulation for the treatment of the visible signs of aging. The formulation and method are not limited to any particular combination of ingredients. The many variations, modifications and alternative applications of the invention that would be apparent to those skilled in the art, and that do not depart from the scope of the invention, are deemed to be covered by the invention.

1.-7. (canceled)

8. At least one mask for the treatment of visible signs of aging comprising:
a first fabric portion saturated with a first skin care formulation for topical application to human skin, wherein the first skin care formulation comprises:
a first exfoliating agent comprising a plant-based B-hydroxy acid;
a first muscle contraction-inhibiting agent comprising an effective amount of acetyl octapeptide-3;
a first anti-inflammatory agent comprising glycosaminoglycans;
a skin repair-activating agent comprising palmitoyl oligopeptide and palmitoyl-tetrapeptide-7; and
carrageenan; and
a second fabric portion saturated with a second skin care formulation for topical application to human skin, wherein the second skin care formulation is different from the first skin care formulation, and wherein the second skin care formulation comprises:
a second exfoliating agent comprising a plant-based B-hydroxy acid;
a second muscle contraction-inhibiting agent comprising an effective amount of acetyl octapeptide-3;
a second anti-inflammatory agent comprising glycosaminoglycans;
an enzyme-inhibiting agent comprising purified rice peptides; and
glycyrrhizinate.

9. The at least one mask according to claim 8, wherein the at least one mask includes a first mask comprising the first fabric portion and a second mask comprising the second fabric portion.

10. The at least one mask of claim 8, wherein the first fabric portion is configured to cover a portion of a human face and the second fabric portion is configured to cover at least one under-eye area of the human face.

11. (canceled)

12. The at least one mask of claim 8, wherein at least one of the first exfoliating agent or the second exfoliating agent comprises willow bark extract.

13. The at least one mask of claim 12, wherein at least one of the first skin care formulation comprises up to 20% by weight of the first exfoliating agent or the second skin care formulation comprises up to 20% by weight of the second exfoliating agent.

14. The at least one mask of claim 12, wherein at least one of the first skin care formulation comprises up to 15% by weight of the first exfoliating agent or the second skin care formulation comprises up to 15% by weight of the second exfoliating agent.

15. The at least one mask of claim 8, wherein at least one of the first skin care formulation comprises up to 10% by weight of the first muscle contraction-inhibiting agent or the second skin care formulation comprises up to 10% by weight of the second muscle contraction-inhibiting agent.

16. The at least one mask of claim 8, wherein at least one of the first skin care formulation comprises up to 5% by weight of the first muscle contraction-inhibiting agent or the second skin care formulation comprises up to 5% by weight of the second muscle contraction-inhibiting agent.

17. The at least one mask of claim 8, wherein at least one of the first skin care formulation comprises up to 10% by weight of the first anti-inflammatory agent or the second skin care formulation comprises up to 10% by weight of the second anti-inflammatory agent.

18. The at least one mask of claim 8, wherein at least one of the first skin care formulation comprises up to 5% by weight of the first anti-inflammatory agent or the second skin care formulation comprises up to 5% by weight of the second anti-inflammatory agent.

19. The at least one mask of claim 8, wherein at least one of the first skin care formulation comprises between 1% and 10% by weight of the first exfoliating agent or the second skin care formulation comprises between 1% and 10% by weight of the second exfoliating agent.

20. The at least one mask of claim 8, wherein the second skin care formulation comprises up to 3% by weight of the second anti-inflammatory agent.

21. The at least one mask of claim 8, wherein at least one of the first skin care formulation or the second skin care formulation comprises a serum.
